(19) United States
(12) Patent Application Publication
(10) Pub. No.: US 2025/0111894 A1
(43) Pub. Date: Apr. 3, 2025

(54) METHOD FOR DIAGNOSING MINIMAL RESIDUAL DISEASE BY DETECTING SEQUENCE OF STRUCTURAL VARIANT IN CFDNA

(71) Applicant: Inha University Research and Business Foundation, Incheon (KR)

(72) Inventor: Hyun Tae SHIN, Incheon (KR)

(73) Assignee: Inha University Research and Business Foundation, Incheon (KR)

(21) Appl. No.: 18/980,382

(22) Filed: Dec. 13, 2024

Related U.S. Application Data
(63) Continuation of application No. PCT/KR2023/008217, filed on Jun. 14, 2023.

(30) Foreign Application Priority Data
Oct. 13, 2022 (KR) 10-2022-0131806

(51) Int. Cl. G16B 20/20; G16B 30/00

(52) U.S. Cl. CPC G16B 20/20; G16B 30/00

(57) ABSTRACT

The present disclosure relates to a system and method for whole genome sequencing (WGS), which detects the sequence of a structural variant with high sensitivity by using cfDNA samples and uses data on the structural variant, to diagnose minimal residual disease in cancer patients. The present disclosure enables detecting the sequence of a structural variant, even when a cancer cell line and NA12878 are mixed at a ratio of 1:12,800, and identifying the sequence of a structural variant in cfDNA of metastatic lung cancer patients. Therefore, the method according to the present disclosure, can be used to detect the sequence of a patient-specific structural variant with high sensitivity, even when a cancer-derived cfDNA is present at a low concentration in blood, etc., and to detect cancer cells remaining in a patient after cancer treatment, even when the sample size is small.

### CROSS-REFERENCE TO RELATED APPLICATIONS

The present application is a continuation of International Patent Application No. PCT/KR2023/008217, filed on Jun. 14, 2023, which is based upon and claims the benefit of priority to Korean Patent Application No. 10-2022-0131806 filed on Oct. 13, 2022. The disclosures of the above-listed applications are hereby incorporated by reference herein in their entirety.

### BACKGROUND

### 1. Technical Field

The present disclosure relates to a novel whole-genome sequencing (WGS) system and a method thereof for detecting a structural variant sequence with high sensitivity in a cell-free DNA (cfDNA) sample derived from a cancer patient and using the same to diagnose minimal residual disease (MRD) in the cancer patient.

The present application claims priority to Korean Patent Application No. 10-2022-0131806, filed on Oct. 13, 2022, the entire contents of which are incorporated herein by reference.

### 2. Description of Related Art

Next Generation Sequencing is a high-throughput genome analysis method that divides the genome into countless fragments, reads them, and aligns the obtained fragments to analyze the genome sequence. Whole-genome sequencing (WGS) using NGS technology is useful for detecting almost all types of somatic variants, and due to this usefulness, it is widely used in various fields, and especially plays a very important role in cancer genomics.

The genome analysis business is rapidly developing worldwide, and this NGS technique is also actively utilized in clinical genomics, pharmaco-genomics, and translational medicine.

Since structural variants (SVs) play an important role in the process of cancer development, many bioinformatics algorithms and tools have been developed to detect somatic structural variants in cancer genomes. Structural variants that occur when specific locations in the genome are cut and combined with each other generate a fusion DNA sequence at the combined site, which is a unique sequence that is not found in normal tissues. It is known that cfDNA is introduced into the bloodstream due to apoptosis in cancer cells, and if a fusion DNA sequence due to a specific structural variant of the primary cancer is detected in cfDNA, it is expected that it can be used to diagnose minimal residual disease (MRD) remaining in a body after cancer treatment.

Targeted high-depth sequencing is widely used as a method to find variants in cfDNA. This method has the advantage of being able to sensitively find variants, but to implement high-depth, a targeted panel suitable for each cancer type needs to be prepared, and an experimental preprocessing process is required to reduce noise.

Therefore, there is a need for a new method that can effectively detect individual cancer-specific structural variant sequences using WGS of cfDNA samples that can be universally utilized to diagnose cancer minimal residual disease.

### SUMMARY

The inventors find a structural variant using WGS of primary cancer to sensitively detect a structural variant sequence in a cfDNA sample, and match the sequence of the structural variant with it in WGS of cfDNA. This enabled the detection of reads having a small number of structural variant sequence information in cfDNA, and the utilization of these for the diagnosis of minimal residual disease in a patient, thereby completing the present disclosure.

In an aspect of the present disclosure, a method for detecting a structural variant sequence in a cfDNA sample derived from a cancer patient includes 1) obtaining consensus structural variant position data of a primary cancer sample that is commonly identified by analyzing a whole genome sequence (WGS) of a cancer tissue derived from a patient with two or more types of structural variant analysis software; and 2) matching a specific sequence generated by a structural variant existing in a cfDNA sample by matching a reference sequence of a structural variant position obtained in step 1) with a cfDNA WGS of the patient and by obtaining a structural variant sequence supporting read existing in the common structural variant position.

In another aspect of the present disclosure, a method for providing information for minimal residual disease (MRD) includes 1) obtaining consensus structural variant position data of a primary cancer sample that is commonly identified by analyzing a whole genome sequence (WGS) of a cancer tissue derived from a patient with two or more types of structural variant analysis software; and 2) matching a specific sequence generated by a structural variant existing in a cfDNA sample by matching a reference sequence of a structural variant position obtained in step 1) with a cfDNA WGS of the patient obtained from the patient after cancer treatment and by obtaining a structural variant sequence supporting read existing in the common structural variant position.

### BRIEF DESCRIPTION OF THE FIGURES

FIG. 1 is a flowchart illustrating a method for detecting a structural variant sequence in cfDNA according to the present disclosure.

FIG. 2 is a schematic diagram illustrating a process of identifying a structural variant sequence which is a comparison of a corresponding reference sequence and each support lead in the second step according to the present disclosure.

FIG. 3 is a diagram illustrating a summary of the result of analytical validation using cell line mixing experiments.

FIGS. 4 and 5 are results illustrating that the structural variant identified in the primary cancer tissue of patient case 1 during the clinical verification experiment are identically identified in cfDNA-1 and cfDNA-2 samples and are not observed in the normal control.

FIG. 6 is a diagram illustrating that KRAS variant is detected (36%) in the primary cancer tissue of patient case 1 and that the same mutation is detected in 19% and 6% in cfDNA-1 and cfDNA-2 samples.

FIGS. 7 and 8 are results illustrating that the structural variant identified in the primary cancer tissue of patient case 2 during the clinical verification experiment are identically identified in cfDNA-1 and cfDNA-2 samples and are not observed in the normal control.

FIG. 9 is a diagram identifying the results of detecting TP53 variants (45%) in the primary cancer tissue of patient case 2, and detecting the same variants in 2% and 0% of cfDNA-1 and cfDNA-2 samples.

FIG. 10 is a diagram identifying the results of detecting SMARCA4 variants (27%) in the primary cancer tissue of patient case 2, and detecting the same variants in 5% and 2% of cfDNA-1 and cfDNA-2 samples.

### DETAILED DESCRIPTION

The present disclosure relates to a method for detecting a structural variant sequence in a cfDNA sample derived from a cancer patient and a method for providing information for minimal residual disease (MRD) using the same.

According to the present disclosure, the structural variant sequence existing in cfDNA of a cancer patient after treatment can be rapidly and sensitively compared and analyzed using a structural variant previously identified in the primary cancer tissue of the cancer patient, thereby monitoring cancer cells remaining in the patient after treatment and providing information for the progress and prognosis of cancer treatment, possibility of recurrence, and the like.

Hereinafter, the present disclosure will be described in detail.

The present disclosure relates to a method for detecting a structural variant sequence in a cfDNA sample derived from a cancer patient including 1) obtaining consensus structural variant position data of a primary cancer sample that is commonly identified by analyzing a whole genome sequence (WGS) of a cancer tissue derived from a patient with two or more types of structural variant analysis software; and 2) matching a specific sequence generated by a structural variant existing in a cfDNA sample by matching a reference sequence of a structural variant position obtained in step 1) with a cfDNA WGS of the patient and by obtaining a structural variant sequence supporting read existing in the common structural variant position.

Step 1) of the present disclosure is a step of obtaining consensus structural variant data of a primary cancer sample, and analyzing WGS of patient-derived cancer tissue and WGS of a normal control group as two or more structural variant analysis data.

In the step, two or more types of structural variant analysis software is used to obtain more accurate patient structural variant data. In this step, WGS and alignment are performed using cancer tissues obtained from the cancer patient, that is, the primary cancer sample and normal controls, and mapping is performed to identify the position of the DNA on the reference genome and the chromosome at which the sequencing reads are located. Once the mapping is complete, the chromosome number and position information for the reference genome are provided for each sequencing read, and the BAM (binary alignment map) format, which is an aligned base fragment containing the information, may be obtained. Using the first structural variant analysis software, ‘structural variant call (SV call)’ is performed to identify the presence of a variant that has a structural variant different from the reference genome sequence at a specific position by comparing and analyzing each sequencing read in the ‘tumor BAM’ file derived from the obtained tumor tissue and the ‘control BAM’ file, and through this, ‘first structural variant position data’ may be obtained.

Next, to obtain the ‘consensus structural variant position data’ of the present disclosure, the structural variant call is performed using the second structural variant analysis software. At this time, to quickly identify the common structural variant, the ‘first structural variant position data’ obtained using the first structural variant analysis software may be input into the second structural variant analysis software to perform the structural variant call.

That is, in the present disclosure, the ‘consensus structural variant position data’ refers to information for the structural variant position that is identified to exist commonly in the SV call through multiple structural variant analysis software.

Step 1) may be used for the purpose of constructing a DB on the structural variant existing in the primary cancer sample of a cancer patient, and the common structural variant position data obtained once in this way may be repeatedly used for monitoring purposes in comparative analysis with the cfDNA structural variant of the same cancer patient obtained at various points in the future.

The structural variant analysis software used in the present disclosure may use various kinds of software known in the art to be capable of detecting somatic structural variant through whole genome analysis without limitation, and for example, two or more kinds selected from the group consisting of DELLY, BRASS, SvABA, dRanger, Pindell, BreakDancer, GASV, Hydra, CNVnator, and JuLI may be used. The two or more kinds of analysis software may be used sequentially, and the first WGS analysis software analyzes each sequencing read through SV call to identify the presence of a variant in which a structural variant different from the standard genome sequence has occurred at a specific position, and provides ‘first structural variant location data’. Afterwards, the first structural variant position data obtained from the first WGS analysis software is input into the second WGS analysis software in VCF, BED format, and the like, and the tumor BAM file to be analyzed is input, so that the ‘common structural variant position data’ that exists in common in the two WGS analysis software may be quickly obtained based on the first structural variant position data that has been identified in advance. The selection of WGS analysis software is not limited thereto, but it is preferable to select the first WGS analysis software that may quickly process a large amount of information, and in the present disclosure, DELLY (Version: 0.8.7, https://github.com/dellytools/delly) is used as a preferred example. In addition, JuLI (https://github.com/sgilab/JuLI, J Mol Diagn. 2020 March; 22(3):304-318), an open source software, is used as the second WGS analysis software that quickly searches for common structural variants based on the first structural variant position data provided by DELLY.

Therefore, step 1) of the present disclosure may be a method for detecting a structural variant in a cfDNA sample derived from a cancer patient, including sequentially performing step 1-1) obtaining first structural variant position data of a primary cancer sample using one type of structural variant analysis software selected from a group consisting of DELLY, BRASS, SvABA, dRanger, Pindell, BreakDancer, GASV, Hydra, and CNVnator; and 1-2) obtaining the consensus structural variant position data of the primary cancer sample that is commonly identified by entering the first structural variant position data obtained in step 1-1) into JuLI.

In addition, the present disclosure includes step 2) matching a specific sequence generated by a structural variant existing in a cfDNA sample by matching a reference sequence of a structural variant position obtained in step 1) with a cfDNA WGS of the patient and by obtaining a structural variant sequence supporting read existing in the common structural variant position.

The present disclosure may detect a small number of structural variants existing in a cfDNA sample with high sensitivity regardless of the cancer type. In the present disclosure, the cfDNA may be cfDNA derived from cerebrospinal fluid, pleural fluid, pericardial fluid, ascites, urine, whole blood, plasma or serum of a cancer patient, but is not limited thereto.

Like the conventional variant detection methods, the detection sensitivity of the present disclosure may be adjusted according to the sequencing depth.

Meanwhile, the cfDNA of the cancer patient in step 2) may be obtained from the patient during or after cancer treatment. In the present disclosure, a DB for common structural variant positions existing in the primary cancer tissue of the cancer patient is constructed in step 1), and then, by comparing the structural variant position sequence in the cfDNA of the patient during or after cancer treatment, information for minute residual cancer cells remaining in the patient after treatment may be provided.

More specifically, step 2) is as follows: First, cfDNA (Cell-free DNA) of the cancer patient is subjected to WGS and alignment, and cfDNA BAM (binary alignment map) format, that is, cfDNA mapping data, in which chromosome number and position information for a standard genome are recorded for each sequencing read obtained. The cfDNA BAM file, which is the cfDNA mapping data obtained thereafter, is input into the structural variant analysis software together with the common structural variant position data obtained in step 1), and a supporting read call is performed to align the supporting reads with the corresponding reference sequence at the common structural variant position identified in step 1), thereby matching the specific sequence caused by the structural variant existing in the cfDNA sample.

The corresponding reference sequence may be used in the same meaning as the standard genome data, and may be hg19 or hg38, or the like.

The method of the present disclosure may be universally used for all cancers regardless of the cancer type, and unlike existing imaging or blood tests, or targeted high-depth sequencing methods, it does not entail a separate process that requires test items or pre-design depending on the cancer type. Therefore, the cancer that is the subject of the present disclosure may include, without limitation, cancer types known in the art, such as gastric cancer, lung cancer, non-small cell lung cancer, breast cancer, ovarian cancer, liver cancer, bronchial cancer, nasopharyngeal cancer, laryngeal cancer, pancreatic cancer, bladder cancer, colon cancer, colon cancer, cervical cancer, bone cancer, non-small cell bone cancer, blood cancer, skin cancer (melanoma, etc.), head or neck cancer, uterine cancer, rectal cancer, anal cancer, colon cancer, fallopian tube cancer, endometrial cancer, vaginal cancer, vulvar cancer, Hodgkin's disease, esophageal cancer, small intestine cancer, endocrine cancer, thyroid cancer, parathyroid cancer, adrenal cancer, soft tissue sarcoma, urethral cancer, penile cancer, prostate cancer, chronic or acute leukemia, lymphocytic lymphoma, kidney or ureteral cancer, renal cell carcinoma, renal pelvic carcinoma, polyploid carcinoma, salivary gland cancer, sarcoma, pseudomyxoma, hepatoblastoma, testicular cancer, glioblastoma, lip cancer, ovarian germ cell tumor, basal cell carcinoma, multiple myeloma, gallbladder cancer, choroidal melanoma, ampulla of Vater, peritoneal cancer, adrenal cancer, tongue cancer, small cell carcinoma, pediatric lymphoma, neuroblastoma, duodenal cancer, ureteral cancer, astrocytoma, meningioma, renal pelvis cancer, vulvar cancer, thymic cancer, central nervous system (CNS) tumor, primary central nervous system lymphoma, spinal cord tumor, brainstem glioma, or pituitary adenoma.

In the present disclosure, the structural variant to be detected may be a somatic structural variant, and may be at least one selected from the group consisting of gene duplication, deletion, translocation, and insertion, and refers to a specific difference in sequence compared to the corresponding reference sequence to be compared. In most cancers, tens to hundreds of structural variants occur in the early stage of cancer development, and these structural variants are maintained as the cancer progresses. In cancer cells, cfDNA flows out into the blood due to apoptosis, and if the structural variant sequence found in the cancer cell is identified in the cfDNA existing in the blood, it can be diagnosed that the cancer remains in the patient's body and that there is a possibility of causing minimal residual disease. Therefore, the structural variant sequence to be detected in the present disclosure is a sequence of a structural variant existing in cfDNA among the structural variants identified to exist in the cancer cell of the patient.

In another aspect of the present disclosure, the present disclosure provides a method for providing information for minimal residual disease (MRD), including: 1) obtaining consensus structural variant position data of a primary cancer sample that is commonly identified by analyzing a whole genome sequence (WGS) of a cancer tissue derived from a patient with two or more types of structural variant analysis software; and 2) matching a specific sequence generated by a structural variant existing in a cfDNA sample by matching a reference sequence of a structural variant position obtained in step 1) with a cfDNA WGS of the patient obtained from the patient after cancer treatment and by obtaining a structural variant sequence supporting read existing in the common structural variant position.

In the present disclosure, the minimal residual disease means a state in which a small number of malignant cells remaining in a patient during or after treatment are molecularly detected. The minimal residual disease is a subject of follow-up observation for various blood cancers and solid cancers, and the patient's responsiveness to treatment may be identified and the risk of recurrence may be predicted by identifying the minimal residual disease. Therefore, the information for the minimal residual disease of the present disclosure may be information for the presence or absence of residual cancer cells during or after treatment, the possibility of cancer recurrence, or the prognosis of cancer treatment. In the case that the method of the present disclosure is performed and the structural variant sequence identified in the primary cancer sample is identified in the patient's cfDNA sample, it may be predicted that the patient has residual cancer cells, has low responsiveness to treatment, has a high risk of recurrence, or has a poor prognosis after cancer treatment.

In the present disclosure, the treatment of cancer patient includes all treatments known in the art, such as radiation therapy, immunotherapy, hormone therapy, chemotherapy, or surgical resection, without limitation.

In addition, the present disclosure provides information for minimal residual disease by comparing the patient's own primary cancer common structural variant with the cfDNA structural variant sequence after treatment, and thus may be utilized as a method for providing patient-tailored information.

Using the present disclosure, the structural variant sequences may be detected with high sensitivity even in blood, plasma or serum derived samples from patients after treatment containing cfDNA of very low purity. In one embodiment of the present disclosure, it is identified that a sufficient number of support leads may be identified even in samples diluted up to 1:12,800, and thus, the structural variant sequence of patient cfDNA samples may be identified to provide information for the minimal residual disease.

The method of detecting the structural variant sequence in a cfDNA sample derived from a cancer patient and the method of providing information for minimal residual disease of the present disclosure may both be methods performed in silico by a computer system. Therefore, the base sequence variation information may be received/obtained through a computer system, and in this respect, the method of the present disclosure may additionally include a step of receiving genetic variation information by a computer system.

The contents of the present disclosure described above are equally applicable to each other unless they are mutually contradictory, and it is also included in the scope of the present disclosure that a person skilled in the art makes appropriate modifications and implements the present disclosure.

The present disclosure is described in detail below through examples, but the scope of the present disclosure is not limited to the following examples.

### Example 1. Construction of a Method for Detecting Structural Variant Specific Sequence in Plasma cfDNA

In order to identify the structural variant specific sequence in plasma cfDNA of a cancer patient, a process including the following two steps is constructed:

### 1. Common (Consensus) Structural Variant of Patient Identification

In order to find common (consensus) structural variant of a patient, two types of analysis tools are used. After performing WGS using patient-derived cancer tissues and control samples, alignment is performed with the default setting of BWA. As an analysis tool, JuLI (https://github.com/sgilab/JuLI), a structural variant detection software that may detect DNA somatic variant and DNA fusion, and DELLY (DELLY Version: 0.8.7, https://github.com/dellytools/delly), another structural variant detection software, are used. First, structural variant calling is performed using the tumor BAM and control BAM file through DELLY to identify the structural variant position (first structural variant position data) present in the patient's primary cancer sample. The identified patient structural variant position result is input into JuLI in BED format, and after inputting the tumor BAM file, structural variant calling is performed to identify the position commonly suspected of structural variant in DELLY and JuLI to obtain consensus structural variant position data. The identified common structural variant position data is then utilized in the form of the JuLI output format in the second step.

2. cfDNA Structural Variant Sequence Detection Through Support Read Detection

After obtaining a plasma sample from a patient after cancer onset, DNA is extracted from the sample, WGS analysis is performed, and alignment is performed with the default setting of BWA to obtain a cfDNA BAM file. The ‘callread’ function of the JuLI program is applied to check whether there is a supporting read indicating a structural variant specific sequence at the same position as the common structural variant position obtained through the first step in the cfDNA BAM file. Using the ‘callread’ function, the specific sequence caused by the structural variant may be matched by comparing each supporting read with a counter reference sequence such as hgl9 or hg38 at the consensus structural variant position. At this time, the specificity may be adjusted by adjusting the default value of splitratio among the JuLI parameters, and the specificity is increased by setting it to 0.95.

Using this method, it is possible to detect with high sensitivity whether there is a cfDNA structural variant support lead having a specific sequence supporting a common structural variant at the fusion site to be identified even with a small number of leads.

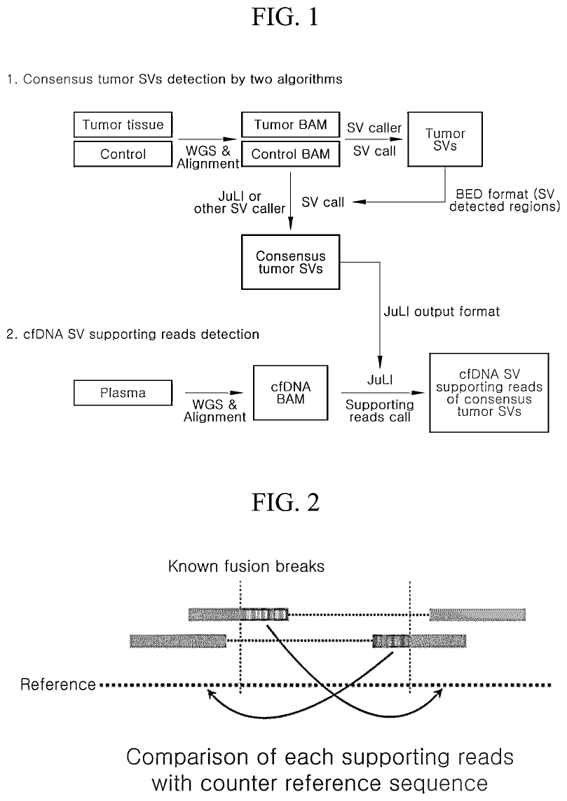

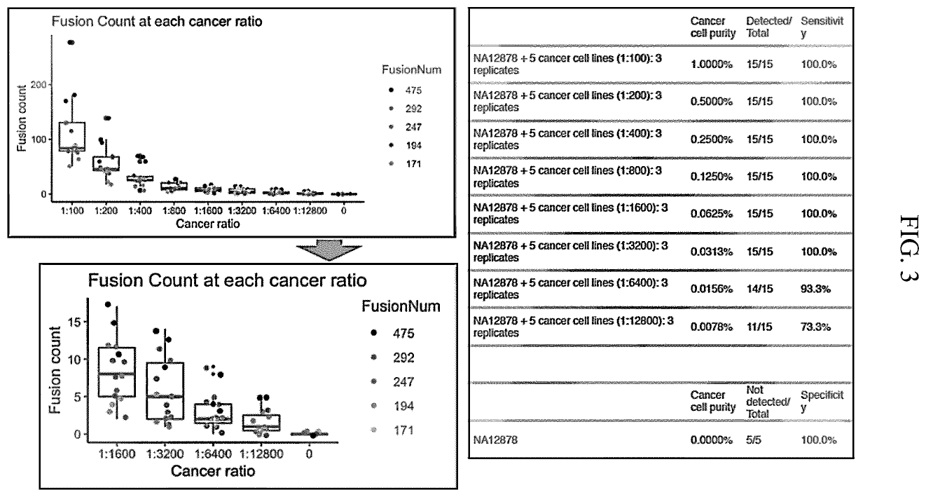

The flow chart of the method for detecting structural variant sequences in plasma cfDNA of the present disclosure, which includes the above two steps, is shown in FIG. 1, and the comparison of the corresponding reference sequence and the support lead in the second step is schematically shown in FIG. 2.

### Experimental Example 1. Analytical Validation

The following describes the result of analytical validation using an actual patient sample and cell lines.

### 1.1 Preparation of Analysis Sample

In order to identify DNA structural variant present in cfDNA, the following sample is prepared. For the analysis verification experiment, cancer cells and standard materials are used as samples. In performing variant definition, NA12878 is designated as a standard material, and structural variants are compared and detected based on this. NA 12878 is purchased from Coriell Institute and used. Five cancer cell lines (WM2664, A375, SNU16, HCC1954, HCC95) are purchased from the Korean Cell Line Bank and used.

DNA of NA12878 and DNA collected from five cancer cell lines (WM2664, A375, SNU16, HCC1954, HCC95) are shared to 150 to 170 bp, which is known as the general cfDNA fragment size. Five types of cancer cell lines are each diluted to 10 ng/ul and mixed in the same volume of 50 ul to prepare a final mixed sample of 10 ng/ul and 250 ul as the initial sample. The initial sample amount of NA 12878 is 50 ng/ul. Thereafter, the mixing ratio of these five types of cancer cell mixed samples and NA 12878 is varied as cell line: NA12878=1:100, 1:200, 1:400, 1:800, 1:1600, 1:3200, 1:6400, and 1:12800, and used in experiments to identify the sensitivity of the present disclosure, and all experiments are performed three times repeatedly.

The patient's clinical sample is obtained from a metastatic lung cancer patient who visited Inha University Hospital, FFPE (Formalin Fixed Paraffin Embedded) cancer tissues, blood buffy coat (control tissue), and plasma sample collected at different time points to collect cfDNA.

### 1.2 Analytical Validation Using Cell Line Mixing Experiments

Analytical validation is performed using the method of Example 1. First, NA 12878 and five cancer cell lines (WM2664, A375, SNU16, HCC1954, HCC95), and their mixed samples are analyzed three times with different mixing ratios (cell line: NA12878=1:100, 1:200, 1:400, 1:800, 1:1600, 1:3200, 1:6400, and 1:12800), and the results are shown in Tables 1 to 8. Each table shows the values of three repetitions of structural variant detection in five cancer cell lines, and the same Sample ID means one BAM file. CD_21_16908 to CD_21_16912 are data corresponding to each cancer cell line as follows: CD_21_16908 (A375SM), CD_21_16909 (HCC95), CD_21_16910 (HCC1954), CD_21_16911 (SNU16) CD_21_16912 (WM2664). Cancer cell lines produced data at an average of 22×, and mixed samples produced data at an average of 70×. Data identifying whether variants in each cell line are detected in WGS using only the reference are shown in Table 9.

TABLE 1

| | 5MIX + | | | Total fusion | cfDNA | |
|---|---|---|---|---|---|---|
| | NA12878 | | | number of each | Fusion | Reads |
| Cell-line | mixing ratio | Sample ID | Remark | cell-line | Number | Count |
| CD_21_16908 | 1:100 | CD_21_17603_CL_D_SRG_1 | WGS_60X - | 171 | 89 | 162 |
| | | | 1st | | | |
| CD_21_16909 | 1:100 | CD_21_17603_CL_D_SRG_1 | WGS_60X - | 292 | 130 | 239 |
| | | | 1st | | | |
| CD_21_16910 | 1:100 | CD_21_17603_CL_D_SRG_1 | WGS_60X - | 475 | 277 | 847 |
| | | | 1st | | | |
| CD_21_16911 | 1:100 | CD_21_17603_CL_D_SRG_1 | WGS_60X - | 194 | 115 | 689 |
| | | | 1st | | | |
| CD_21_16912 | 1:100 | CD_21_17603_CL_D_SRG_1 | WGS_60X - | 247 | 131 | 251 |
| | | | 1st | | | |
| CD_21_16908 | 1:100 | CD_22_06205_CL_D_SRG_1 | WGS_60X - | 171 | 51 | 87 |
| | | | 2nd | | | |
| CD_21_16909 | 1:100 | CD_22_06205_CL_D_SRG_1 | WGS_60X - | 292 | 84 | 158 |
| | | | 2nd | | | |
| CD_21_16910 | 1:100 | CD_22_06205_CL_D_SRG_1 | WGS_60X - | 475 | 181 | 407 |
| | | | 2nd | | | |
| CD_21_16911 | 1:100 | CD_22_06205_CL_D_SRG_1 | WGS_60X - | 194 | 80 | 382 |
| | | | 2nd | | | |
| CD_21_16912 | 1:100 | CD_22_06205_CL_D_SRG_1 | WGS_60X - | 247 | 79 | 118 |
| | | | 2nd | | | |
| CD_21_16908 | 1:100 | CD_22_10086_CL_D_SRG_1 | WGS_60X - | 171 | 64 | 110 |
| | | | 3rd | | | |
| CD_21_16909 | 1:100 | CD_22_10086_CL_D_SRG_1 | WGS_60X - | 292 | 83 | 137 |
| | | | 3rd | | | |
| CD_21_16910 | 1:100 | CD_22_10086_CL_D_SRG_1 | WGS_60X - | 475 | 170 | 442 |
| | | | 3rd | | | |
| CD_21_16911 | 1:100 | CD_22_10086_CL_D_SRG_1 | WGS_60X - | 194 | 76 | 396 |
| | | | 3rd | | | |
| CD_21_16912 | 1:100 | CD_22_10086_CL_D_SRG_1 | WGS_60X - | 247 | 79 | 109 |
| | | | 3rd | | | |

TABLE 2

| | 5MIX + | | | Total fusion | cfDNA | |
|---|---|---|---|---|---|---|
| | NA12878 | | | number of each | Fusion | Reads |
| Cell-line | mixing ratio | Sample ID | Remark | cell-line | Number | Count |
| CD_21_16908 | 1:200 | CD_21_17605_CL_D_SRG_1 | WGS_60X - | 171 | 18 | 23 |
| | | | 1st | | | |
| CD_21_16909 | 1:200 | CD_21_17605_CL_D_SRG_1 | WGS_60X - | 292 | 49 | 71 |
| | | | 1st | | | |
| CD_21_16910 | 1:200 | CD_21_17605_CL_D_SRG_1 | WGS_60X - | 475 | 100 | 197 |
| | | | 1st | | | |
| CD_21_16911 | 1:200 | CD_21_17605_CL_D_SRG_1 | WGS_60X - | 194 | 45 | 194 |
| | | | 1st | | | |
| CD_21_16912 | 1:200 | CD_21_17605_CL_D_SRG_1 | WGS_60X - | 247 | 46 | 71 |
| | | | 1st | | | |
| CD_21_16908 | 1:200 | CD_22_06207_CL_D_SRG_1 | WGS_60X - | 171 | 23 | 38 |
| | | | 2nd | | | |
| CD_21_16909 | 1:200 | CD_22_06207_CL_D_SRG_1 | WGS_60X - | 292 | 45 | 60 |
| | | | 2nd | | | |
| CD_21_16910 | 1:200 | CD_22_06207_CL_D_SRG_1 | WGS_60X - | 475 | 94 | 192 |
| | | | 2nd | | | |
| CD_21_16911 | 1:200 | CD_22_06207_CL_D_SRG_1 | WGS_60X - | 194 | 44 | 182 |
| | | | 2nd | | | |
| CD_21_16912 | 1:200 | CD_22_06207_CL_D_SRG_1 | WGS_60X - | 247 | 38 | 56 |
| | | | 2nd | | | |
| CD_21_16908 | 1:200 | CD_22_10087_CL_D_SRG_1 | WGS_60X - | 171 | 42 | 64 |
| | | | 3rd | | | |
| CD_21_16909 | 1:200 | CD_22_10087_CL_D_SRG_1 | WGS_60X - | 292 | 60 | 86 |
| | | | 3rd | | | |
| CD_21_16910 | 1:200 | CD_22_10087_CL_D_SRG_1 | WGS_60X - | 475 | 139 | 290 |
| | | | 3rd | | | |
| CD_21_16911 | 1:200 | CD_22_10087_CL_D_SRG_1 | WGS_60X - | 194 | 67 | 299 |
| | | | 3rd | | | |
| CD_21_16912 | 1:200 | CD_22_10087_CL_D_SRG_1 | WGS_60X - | 247 | 69 | 108 |
| | | | 3rd | | | |

TABLE 3

| | 5MIX + | | | Total fusion | cfDNA | |
|---|---|---|---|---|---|---|
| | NA12878 | | | number of each | Fusion | Reads |
| Cell-line | mixing ratio | Sample ID | Remark | cell-line | Number | Count |
| CD_21_16908 | 1:400 | CD_21_17606_CL_D_SRG_1 | WGS_60X - | 171 | 7 | 11 |
| | | | 1st | | | |
| CD_21_16909 | 1:400 | CD_21_17606_CL_D_SRG_1 | WGS_60X - | 292 | 34 | 41 |
| | | | 1st | | | |
| CD_21_16910 | 1:400 | CD_21_17606_CL_D_SRG_1 | WGS_60X - | 475 | 60 | 108 |
| | | | 1st | | | |
| CD_21_16911 | 1:400 | CD_21_17606_CL_D_SRG_1 | WGS_60X - | 194 | 30 | 92 |
| | | | 1st | | | |
| CD_21_16912 | 1:400 | CD_21_17606_CL_D_SRG_1 | WGS_60X - | 247 | 25 | 32 |
| | | | 1st | | | |
| CD_21_16908 | 1:400 | CD_22_06208_CL_D_SRG_1 | WGS_60X - | 171 | 14 | 18 |
| | | | 2nd | | | |
| CD_21_16909 | 1:400 | CD_22_06208_CL_D_SRG_1 | WGS_60X - | 292 | 24 | 27 |
| | | | 2nd | | | |
| CD_21_16910 | 1:400 | CD_22_06208_CL_D_SRG_1 | WGS_60X - | 475 | 68 | 113 |
| | | | 2nd | | | |
| CD_21_16911 | 1:400 | CD_22_06208_CL_D_SRG_1 | WGS_60X - | 194 | 30 | 91 |
| | | | 2nd | | | |
| CD_21_16912 | 1:400 | CD_22_06208_CL_D_SRG_1 | WGS_60X - | 247 | 25 | 35 |
| | | | 2nd | | | |
| CD_21_16908 | 1:400 | CD_22_10088_CL_D_SRG_1 | WGS_60X - | 171 | 16 | 23 |
| | | | 3rd | | | |
| CD_21_16909 | 1:400 | CD_22_10088_CL_D_SRG_1 | WGS_60X - | 292 | 26 | 36 |
| | | | 3rd | | | |
| CD_21_16910 | 1:400 | CD_22_10088_CL_D_SRG_1 | WGS_60X - | 475 | 70 | 130 |
| | | | 3rd | | | |
| CD_21_16911 | 1:400 | CD_22_10088_CL_D_SRG_1 | WGS_60X - | 194 | 26 | 114 |
| | | | 3rd | | | |
| CD_21_16912 | 1:400 | CD_22_10088_CL_D_SRG_1 | WGS_60X - | 247 | 28 | 38 |
| | | | 3rd | | | |

TABLE 4

| | 5MIX + | | | Total fusion | cfDNA | |
|---|---|---|---|---|---|---|
| | NA12878 | | | number of each | Fusion | Reads |
| Cell-line | mixing ratio | Sample ID | Remark | cell-line | Number | Count |
| CD_21_16908 | 1:800 | CD_21_17607_CL_D_SRG_1 | WGS_60X - | 171 | 5 | 11 |
| | | | 1st | | | |
| CD_21_16909 | 1:800 | CD_21_17607_CL_D_SRG_1 | WGS_60X - | 292 | 7 | 9 |
| | | | 1st | | | |
| CD_21_16910 | 1:800 | CD_21_17607_CL_D_SRG_1 | WGS_60X - | 475 | 20 | 35 |
| | | | 1st | | | |
| CD_21_16911 | 1:800 | CD_21_17607_CL_D_SRG_1 | WGS_60X - | 194 | 11 | 33 |
| | | | 1st | | | |
| CD_21_16912 | 1:800 | CD_21_17607_CL_D_SRG_1 | WGS_60X - | 247 | 9 | 10 |
| | | | 1st | | | |
| CD_21_16908 | 1:800 | CD_22_06209_CL_D_SRG_1 | WGS_60X - | 171 | 6 | 8 |
| | | | 2nd | | | |
| CD_21_16909 | 1:800 | CD_22_06209_CL_D_SRG_1 | WGS_60X - | 292 | 13 | 18 |
| | | | 2nd | | | |
| CD_21_16910 | 1:800 | CD_22_06209_CL_D_SRG_1 | WGS_60X - | 475 | 27 | 49 |
| | | | 2nd | | | |
| CD_21_16911 | 1:800 | CD_22_06209_CL_D_SRG_1 | WGS_60X - | 194 | 21 | 68 |
| | | | 2nd | | | |
| CD_21_16912 | 1:800 | CD_22_06209_CL_D_SRG_1 | WGS_60X - | 247 | 12 | 15 |
| | | | 2nd | | | |
| CD_21_16908 | 1:800 | CD_22_10089_CL_D_SRG_1 | WGS_60X - | 171 | 5 | 9 |
| | | | 3rd | | | |
| CD_21_16909 | 1:800 | CD_22_10089_CL_D_SRG_1 | WGS_60X - | 292 | 11 | 17 |
| | | | 3rd | | | |
| CD_21_16910 | 1:800 | CD_22_10089_CL_D_SRG_1 | WGS_60X - | 475 | 27 | 49 |
| | | | 3rd | | | |
| CD_21_16911 | 1:800 | CD_22_10089_CL_D_SRG_1 | WGS_60X - | 194 | 21 | 65 |
| | | | 3rd | | | |
| CD_21_16912 | 1:800 | CD_22_10089_CL_D_SRG_1 | WGS_60X - | 247 | 9 | 9 |
| | | | 3rd | | | |

TABLE 5

| | 5MIX + | | | Total fusion | cfDNA | |
|---|---|---|---|---|---|---|
| | NA12878 | | | number of each | Fusion | Reads |
| Cell-line | mixing ratio | Sample ID | Remark | cell-line | Number | Count |
| CD_21_16908 | 1:1600 | CD_21_17604_CL_D_SRG_1 | WGS_60X - | 171 | 4 | 4 |
| | | | 1st | | | |
| CD_21_16909 | 1:1600 | CD_21_17604_CL_D_SRG_1 | WGS_60X - | 292 | 8 | 10 |
| | | | 1st | | | |
| CD_21_16910 | 1:1600 | CD_21_17604_CL_D_SRG_1 | WGS_60X - | 475 | 11 | 23 |
| | | | 1st | | | |
| CD_21_16911 | 1:1600 | CD_21_17604_CL_D_SRG_1 | WGS_60X - | 194 | 8 | 29 |
| | | | 1st | | | |
| CD_21_16912 | 1:1600 | CD_21_17604_CL_D_SRG_1 | WGS_60X - | 247 | 6 | 9 |
| | | | 1st | | | |
| CD_21_16908 | 1:1600 | CD_22_06206_CL_D_SRG_1 | WGS_60X - | 171 | 5 | 5 |
| | | | 2nd | | | |
| CD_21_16909 | 1:1600 | CD_22_06206_CL_D_SRG_1 | WGS_60X - | 292 | 2 | 2 |
| | | | 2nd | | | |
| CD_21_16910 | 1:1600 | CD_22_06206_CL_D_SRG_1 | WGS_60X - | 475 | 15 | 23 |
| | | | 2nd | | | |
| CD_21_16911 | 1:1600 | CD_22_06206_CL_D_SRG_1 | WGS_60X - | 194 | 12 | 31 |
| | | | 2nd | | | |
| CD_21_16912 | 1:1600 | CD_22_06206_CL_D_SRG_1 | WGS_60X - | 247 | 10 | 11 |
| | | | 2nd | | | |
| CD_21_16908 | 1:1600 | CD_22_10090_CL_D_SRG_1 | WGS_60X - | 171 | 3 | 3 |
| | | | 3rd | | | |
| CD_21_16909 | 1:1600 | CD_22_10090_CL_D_SRG_1 | WGS_60X - | 292 | 5 | 6 |
| | | | 3rd | | | |
| CD_21_16910 | 1:1600 | CD_22_10090_CL_D_SRG_1 | WGS_60X - | 475 | 17 | 26 |
| | | | 3rd | | | |
| CD_21_16911 | 1:1600 | CD_22_10090_CL_D_SRG_1 | WGS_60X - | 194 | 12 | 27 |
| | | | 3rd | | | |
| CD_21_16912 | 1:1600 | CD_22_10090_CL_D_SRG_1 | WGS_60X - | 247 | 10 | 11 |
| | | | 3rd | | | |

TABLE 6

| | 5MIX + | | | Total fusion | cfDNA | |
|---|---|---|---|---|---|---|
| | NA12878 | | | number of each | Fusion | Reads |
| Cell-line | mixing ratio | Sample ID | Remark | cell-line | Number | Count |
| CD_21_16908 | 1:3200 | CD_22_10091_CL_D_SRG_1 | WGS_60_high | 171 | 5 | 5 |
| | | | dilution - 1st | | | |
| CD_21_16909 | 1:3200 | CD_22_10091_CL_D_SRG_1 | WGS_60_high | 292 | 2 | 2 |
| | | | dilution - 1st | | | |
| CD_21_16910 | 1:3200 | CD_22_10091_CL_D_SRG_1 | WGS_60_high | 475 | 9 | 12 |
| | | | dilution - 1st | | | |
| CD_21_16911 | 1:3200 | CD_22_10091_CL_D_SRG_1 | WGS_60_high | 194 | 10 | 14 |
| | | | dilution - 1st | | | |
| CD_21_16912 | 1:3200 | CD_22_10091_CL_D_SRG_1 | WGS_60_high | 247 | 3 | 5 |
| | | | dilution - 1st | | | |
| CD_21_16908 | 1:3200 | CD_22_10094_CL_D_SRG_1 | WGS_60_high | 171 | 2 | 3 |
| | | | dilution - 2nd | | | |
| CD 21_16909 | 1:3200 | CD_22_10094_CL_D_SRG_1 | WGS_60_high | 292 | 7 | 9 |
| | | | dilution - 2nd | | | |
| CD_21_16910 | 1:3200 | CD_22_10094_CL_D_SRG_1 | WGS_60_high | 475 | 14 | 19 |
| | | | dilution - 2nd | | | |
| CD_21_16911 | 1:3200 | CD_22_10094_CL_D_SRG_1 | WGS_60_high | 194 | 11 | 19 |
| | | | dilution - 2nd | | | |
| CD_21_16912 | 1:3200 | CD_22_10094_CL_D_SRG_1 | WGS_60_high | 247 | 4 | 5 |
| | | | dilution - 2nd | | | |
| CD 21_16908 | 1:3200 | CD_22_10097_CL_D_SRG_1 | WGS_60_high | 171 | 1 | 1 |
| | | | dilution - 3rd | | | |
| CD_21_16909 | 1:3200 | CD_22_10097_CL_D_SRG_1 | WGS_60_high | 292 | 2 | 2 |
| | | | dilution - 3rd | | | |
| CD_21_16910 | 1:3200 | CD_22_10097_CL_D_SRG_1 | WGS_60_high | 475 | 13 | 17 |
| | | | dilution - 3rd | | | |
| CD_21_16911 | 1:3200 | CD_22_10097_CL_D_SRG_1 | WGS_60_high | 194 | 5 | 7 |
| | | | dilution - 3rd | | | |
| CD_21_16912 | 1:3200 | CD_22_10097_CL_D_SRG_1 | WGS_60_high | 247 | 1 | 1 |
| | | | dilution - 3rd | | | |

TABLE 7

| | 5MIX + | | | Total fusion | cfDNA | |
|---|---|---|---|---|---|---|
| | NA12878 | | | number of each | Fusion | Reads |
| Cell-line | mixing ratio | Sample ID | Remark | cell-line | Number | Count |
| CD_21_16908 | 1:6400 | CD_22_10092_CL_D_SRG_1 | WGS_60_high | 171 | 2 | 2 |
| | | | dilution - 1st | | | |
| CD_21_16909 | 1:6400 | CD_22_10092_CL_D_SRG_1 | WGS_60_high | 292 | 0 | 0 |
| | | | dilution - 1st | | | |
| CD_21_16910 | 1:6400 | CD_22 10092_CL_D_SRG_1 | WGS_60_high | 475 | 3 | 4 |
| | | | dilution - 1st | | | |
| CD_21_16911 | 1:6400 | CD_22_10092_CL_D_SRG_1 | WGS_60_high | 194 | 9 | 12 |
| | | | dilution - 1st | | | |
| CD_21_16912 | 1:6400 | CD_22_10092_CL_D_SRG_1 | WGS_60_high | 247 | 1 | 1 |
| | | | dilution - 1st | | | |
| CD_21_16908 | 1:6400 | CD_22_10095_CL_D_SRG_1 | WGS_60_high | 171 | 1 | 2 |
| | | | dilution - 2nd | | | |
| CD_21_16909 | 1:6400 | CD_22_10095_CL_D_SRG_1 | WGS_60_high | 292 | 2 | 2 |
| | | | dilution - 2nd | | | |
| CD_21_16910 | 1:6400 | CD_22_10095_CL_D_SRG_1 | WGS_60_high | 475 | 8 | 13 |
| | | | dilution - 2nd | | | |
| CD_21_16911 | 1:6400 | CD_22_10095_CL_D_SRG_1 | WGS_60_high | 194 | 2 | 2 |
| | | | dilution - 2nd | | | |
| CD_21_16912 | 1:6400 | CD_22_10095_CL_D_SRG_1 | WGS_60_high | 247 | 1 | 1 |
| | | | dilution - 2nd | | | |
| CD_21_16908 | 1:6400 | CD_22_10098_CL_D_SRG_1 | WGS_60_high | 171 | 2 | 3 |
| | | | dilution - 3rd | | | |
| CD_21_16909 | 1:6400 | CD_22_10098_CL_D_SRG_1 | WGS_60_high | 292 | 4 | 5 |
| | | | dilution - 3rd | | | |
| CD_21_16910 | 1:6400 | CD_22_10098_CL_D_SRG_1 | WGS_60_high | 475 | 4 | 8 |
| | | | dilution - 3rd | | | |
| CD_21_16911 | 1:6400 | CD_22_10098_CL_D_SRG_1 | WGS_60_high | 194 | 5 | 12 |
| | | | dilution - 3rd | | | |
| CD_21_16912 | 1:6400 | CD_22_10098_CL_D_SRG_1 | WGS_60_high | 247 | 4 | 4 |
| | | | dilution - 3rd | | | |

TABLE 8

| | 5MIX + | | | Total fusion | cfDNA | |
|---|---|---|---|---|---|---|
| | NA12878 | | | number of each | Fusion | Reads |
| Cell-line | mixing ratio | Sample ID | Remark | cell-line | Number | Count |
| CD_21_16908 | 1:12800 | CD_22_10093_CL_D_SRG_1 | WGS_60_high | 171 | 2 | 2 |
| | | | dilution - 1st | | | |
| CD_21_16909 | 1:12800 | CD_22_10093_CL_D_SRG_1 | WGS_60_high | 292 | 0 | 0 |
| | | | dilution - 1st | | | |
| CD_21_16910 | 1:12800 | CD_22_10093_CL_D_SRG_1 | WGS_60_high | 475 | 5 | 8 |
| | | | dilution - 1st | | | |
| CD_21_16911 | 1:12800 | CD_22_10093_CL_D_SRG_1 | WGS_60_high | 194 | 3 | 3 |
| | | | dilution - 1st | | | |
| CD_21_16912 | 1:12800 | CD_22_10093_CL_D_SRG_1 | WGS_60_high | 247 | 0 | 0 |
| | | | dilution - 1st | | | |
| CD_21_16908 | 1:12800 | CD_22_10096_CL_D_SRG_1 | WGS_60_high | 171 | 2 | 2 |
| | | | dilution - 2nd | | | |
| CD_21_16909 | 1:12800 | CD_22_10096_CL_D_SRG_1 | WGS_60_high | 292 | 1 | 1 |
| | | | dilution - 2nd | | | |
| CD_21_16910 | 1:12800 | CD_22_10096_CL_D_SRG_1 | WGS_60_high | 475 | 1 | 2 |
| | | | dilution - 2nd | | | |
| CD_21_16911 | 1:12800 | CD_22_10096_CL_D_SRG_1 | WGS_60_high | 194 | 3 | 4 |
| | | | dilution - 2nd | | | |
| CD_21_16912 | 1:12800 | CD_22_10096_CL_D_SRG_1 | WGS_60_high | 247 | 1 | 1 |
| | | | dilution - 2nd | | | |
| CD_21_16908 | 1:12800 | CD_22_10099_CL_D_SRG_1 | WGS_60_high | 171 | 0 | 0 |
| | | | dilution - 3rd | | | |
| CD_21_16909 | 1:12800 | CD_22_10099_CL_D_SRG_1 | WGS_60_high | 292 | 1 | 2 |
| | | | dilution - 3rd | | | |
| CD_21_16910 | 1:12800 | CD_22_10099_CL_D_SRG_1 | WGS_60_high | 475 | 5 | 5 |
| | | | dilution - 3rd | | | |
| CD_21_16911 | 1:12800 | CD_22_10099_CL_D_SRG_1 | WGS_60_high | 194 | 0 | 0 |
| | | | dilution - 3rd | | | |
| CD_21_16912 | 1:12800 | CD_22_10099_CL_D_SRG_1 | WGS_60_high | 247 | 1 | 1 |
| | | | dilution - 3rd | | | |

TABLE 9

| | | | Total | | |
|---|---|---|---|---|---|
| | | | fusion | | |
| | | | number | cfDNA | |
| | | | of each | Fusion | Reads |
| Cell-line | reference | Sample ID | cell-line | Number | Count |
| CD_21_ | NA12878 | CD_22_06210_ | 171 | 0 | 0 |
| 16908 | | ET_D_SRG_1 | | | |
| CD_21_ | NA12878 | CD_22_06210_ | 292 | 0 | 0 |
| 16909 | | ET_D_SRG_1 | | | |
| CD_21_ | NA12878 | CD_22_06210_ | 475 | 0 | 0 |
| 16910 | | ET_D_SRG_1 | | | |
| CD_21_ | NA12878 | CD_22_06210_ | 194 | 0 | 0 |
| 16911 | | ET_D_SRG_1 | | | |
| CD_21_ | NA12878 | CD_22_06210_ | 247 | 0 | 0 |
| 16912 | | ET_D_SRG_1 | | | |

The ‘Total fusion number of each cell-line (corresponding to the structural variant of the primary cancer sample in Example 1)’, which indicates the number of structural variants present in cancer cell lines, is 171, 292, 475, 194, and 247, respectively, after removing germline structural variants that are duplicated in other cell lines in each cancer type. As shown in Tables 1 to 8, the structural variants present in each cancer cell line are also detected in the cfDNA BAM file (cfDNA Fusion Number), and the supporting reads supporting this are identified (reads count). In addition, it is identified that the structural variant sequence of each cell line is not detected in the reference WGS where the cell lines are not mixed (Table 9).

The results summarizing the sensitivity and specificity of the experiments using cell lines are shown in Table 10 and FIG. 3.

TABLE 10

| | Cancer | Detected/ | |
|---|---|---|---|
| | cell purity | Total | Sensitivity |
| NA12878 + 5 cancer | 1.0000% | 15/15 | 100.0% |
| cell lines (1:100): | | | |
| 3 replicates | | | |
| NA12878 + 5 cancer | 0.5000% | 15/15 | 100.0% |
| cell lines (1:200): | | | |
| 3 replicates | | | |
| NA12878 + 5 cancer | 0.2500% | 15/15 | 100.0% |
| cell lines (1:400): | | | |
| 3 replicates | | | |
| NA12878 + 5 cancer | 0.1250% | 15/15 | 100.0% |
| cell lines (1:800): | | | |
| 3 replicates | | | |
| NA12878 + 5 cancer | 0.0625% | 15/15 | 100.0% |
| cell lines (1:1600): | | | |
| 3 replicates | | | |
| NA12878 + 5 cancer | 0.0313% | 15/15 | 100.0% |
| cell lines (1:3200): | | | |
| 3 replicates | | | |
| NA12878 + 5 cancer | 0.0156% | 14/15 | 93.3% |
| cell lines (1:6400): | | | |
| 3 replicates | | | |
| NA12878 + 5 cancer | 0.0078% | 11/15 | 73.3% |
| cell lines (1:12800): | | | |
| 3 replicates | | | |
| | | Not | |
| | Cancer | detected/ | |
| | cell purity | Total | Specificity |
| NA12878 | 0.0000% | 5/5 | 100.0% |

As summarized in Table 10, the number of support leads tended to decrease gradually as the dilution ratio increased, but it is identified that support sequences capable of identifying structural variant sequences are identified even when diluted up to 1:12,800, and that detection is possible with a high sensitivity of approximately 73.3%. Particularly, in the case of HCC1954, which has 475 structural variants, 100% detection is possible even when diluted to 1:12,800. This means that the method of the present disclosure may very effectively detect structural variant sequence present in cfDNA samples, providing information for minimal residual disease in cancer patients.

### 1.3 Clinical Validation Using Clinical Sample

A clinical validation experiment is performed to determine whether the method of the present disclosure may effectively detect variants in actual patient blood and cfDNA.

The samples are the patient's primary cancer tissue, blood, and cfDNA1 and cfDNA2 obtained at two or more different time points, and the same analysis method as Example 1 is used.

Each cancer patient is a metastatic lung cancer patient, and are described as case 1 and case 2, respectively. Approximately 30×WGS data is generated using FFPE cancer tissue obtained from the patient, and an average of 48×WGS data is generated from the remaining cfDNA samples.

Specifically, WGS analysis is performed using DELLY using the patient's FFPE cancer tissue as a sample, and alignment is performed with the default setting of BWA, and common (consensus) structural variants of the patient commonly identified in the two analysis tools are identified using JuLI. This is utilized as the JuLI output format. Thereafter, the cfDNA structural variant sequence is identified through a two-step supporting read call that compares the BAM file obtained by WGS of plasma cfDNA samples obtained at different time points and the consensus structural variant data identified above with the corresponding reference sequence. This allows for more sensitive detection of cfDNA structural variants present in the patient's plasma obtained at different time points, thereby enabling diagnosis of residual cancer in the patient.

### Patient Case 1

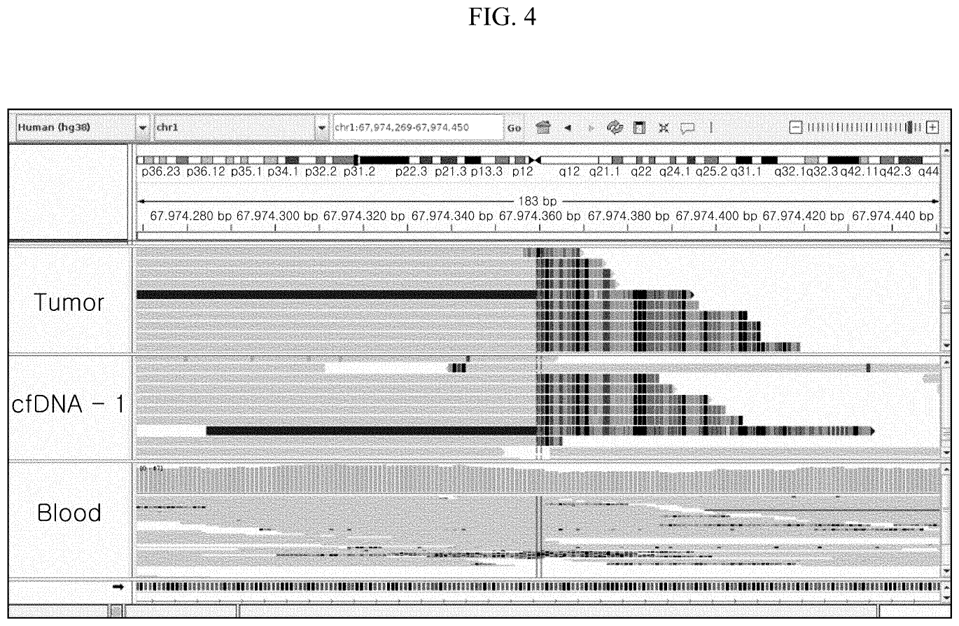

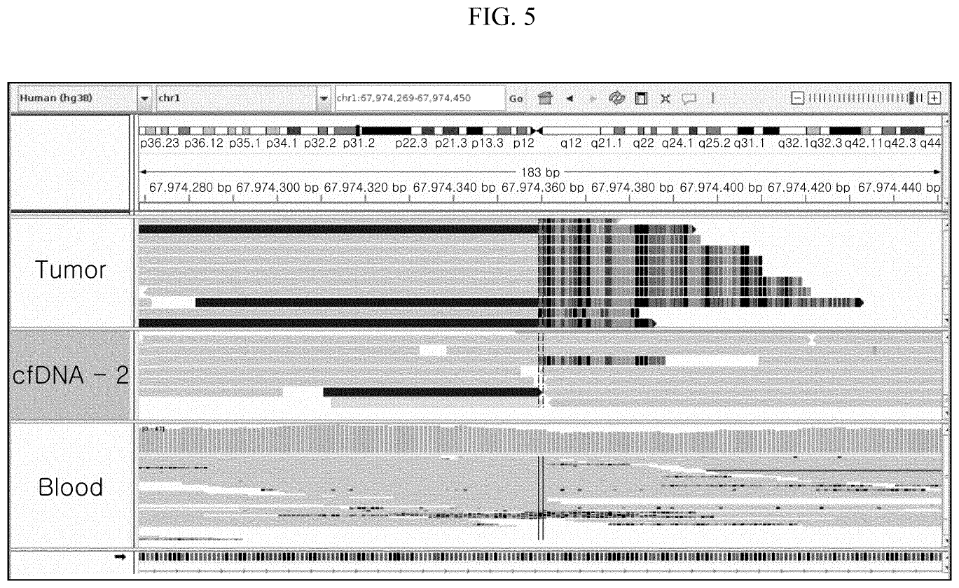

FIGS. 4 and 5 are the analysis results of Case 1, and show the results of identifying the structural variant sequence using cfDNA obtained at different time points, 2006.11.07 and 2007.04.12, respectively. In FIGS. 4 and 5, it is identified that the structural variants identified in the patient-derived primary cancer tissue are identically identified in the cfDNA-1 and cfDNA-2 samples.

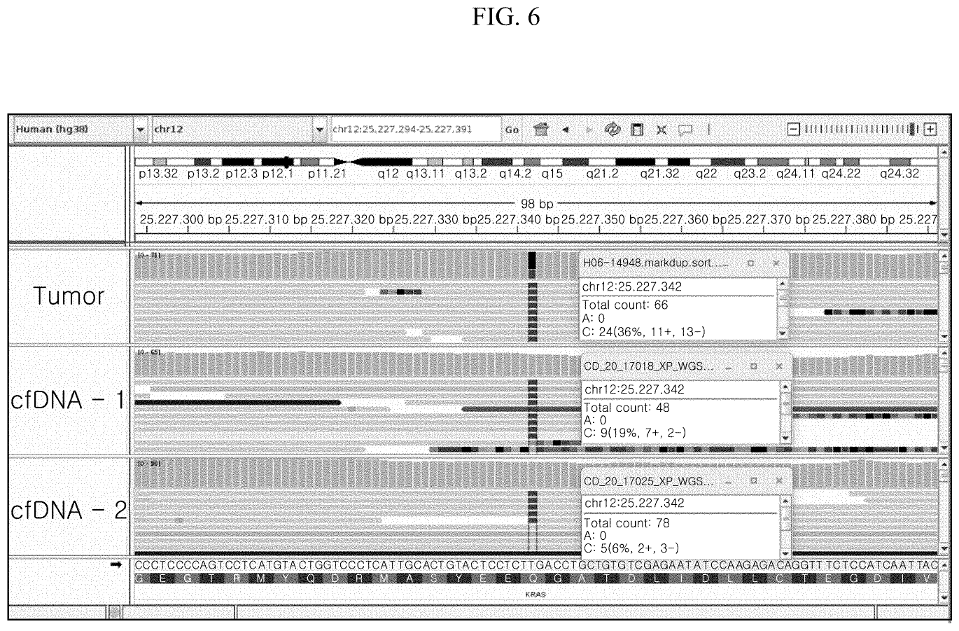

In order to verify the effect of the present disclosure, panel sequencing is performed on the patient's primary cancer to identify cancer variants, which are then identified in cfDNA. Through panel sequencing, it is identified that case 1 patient is a patient with approximately 30% KRAS variants, and the same variants are actually identified in WGS of the primary cancer, cfDNA-1, and cfDNA-2. The results of identifying the position of the KRAS variant in WGS are shown in FIG. 6.

In FIG. 6, it is identified that KRAS variants are detected at a similar level (36%) as in panel sequencing in primary cancer tissues, and KRAS variants are detected at 19% and 6% in cfDNA-1 and cfDNA-2 samples, respectively, verifying that the method of the present disclosure may identify microscopic residual cancer through structural variant sequence analysis of cfDNA samples. The number of read counts detected in the corresponding patient is as shown in Table 11 below.

TABLE 11

| | Tumor | cfDNA | |
|---|---|---|---|
| cfDNA | Fusion | Fusion | |
| samples | Number | Number | ReadsCount |
| cfDNA-1 | 131 | 115 | 4122 |
| cfDNA-2 | 131 | 112 | 1926 |

According to Table 11, patient case 1 is a patient with a total of 131 structural variant sequences, which are fusion sequences, as analyzed from primary cancer samples. Through the method of the present disclosure, 115 or 112 structural variants are identified in cfDNA, and 4122 supporting reads are identified in the cfDNA-1 sample showing 19% KRAS variant, and 1926 supporting reads are identified in the cfDNA-2 sample showing 6% KRAS variant.

This is a result showing that the present disclosure has a much better detection ability, as it shows more than 1900 reads even at about 6% variant, compared to the conventional method of analyzing plasma cfDNA with WGS, which shows low sensitivity that makes it difficult to detect variants of 5% or less.

### Patient Case 2

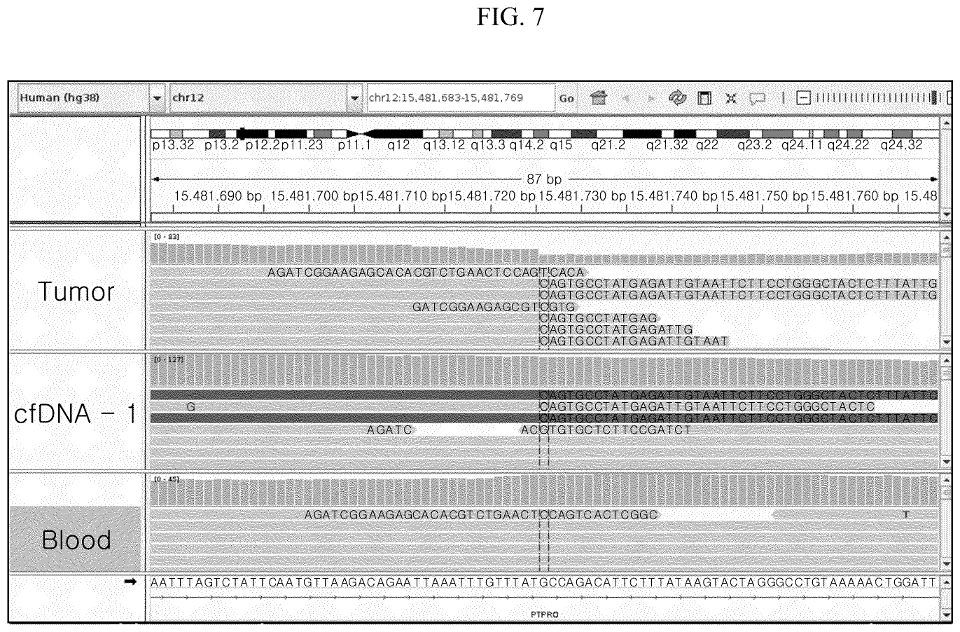

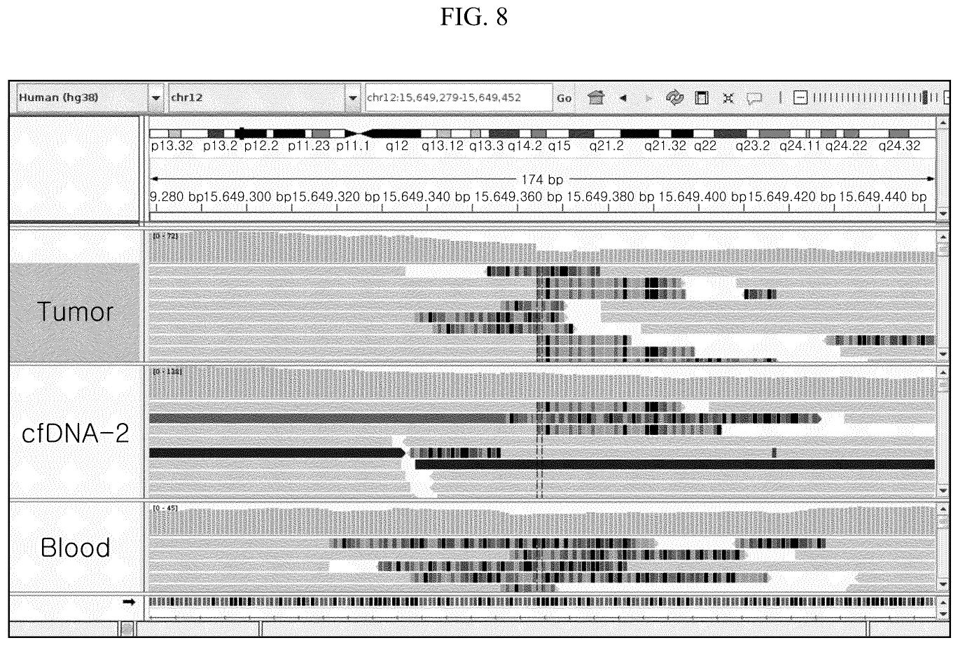

FIGS. 7 and 8 are the analysis results of Case 2, and show the results of identifying the structural variant sequence using cfDNA obtained at different times, 2010.10.29 and 2011.01.04, as samples. FIGS. 7 and 8 show that the structural variants identified in the patient-derived primary cancer tissue are also identified in the cfDNA-1 and cfDNA-2 samples.

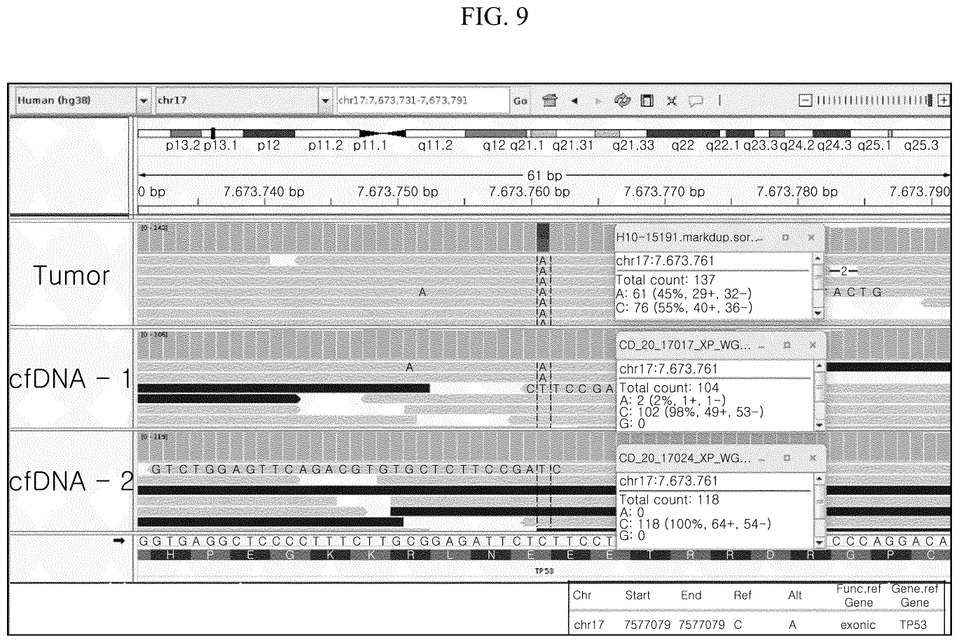

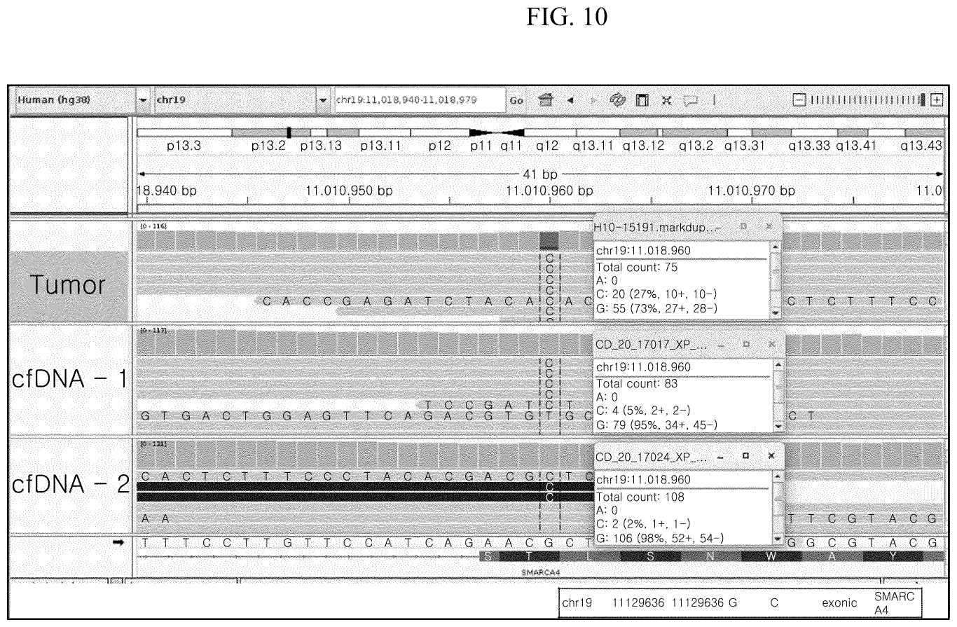

Through panel sequencing, the variants in Case 2 patient are actually identified in the WGS of the primary cancer, cfDNA-1, and cfDNA-2, and this is shown in FIGS. 9 and 10.

In FIG. 9, 2% and 0% of TP53 variants are identified in cfDNA-1 and cfDNA-2, respectively, and 5% and 2% of SMARCA4 variants are identified in FIG. 10, but as shown in Table 12, 128 reads are identified in cfDNA-1 showing 2-5% variants, and 118 reads are identified in 0-2% variants, identifying that sufficient support reads indicating the presence of structural variants are detected even in samples showing low allele frequencies of 2 to 5% or less.

TABLE 12

| | Tumor | cfDNA | |
|---|---|---|---|
| cfDNA | Fusion | Fusion | Reads |
| samples | Number | Number | Count |
| cfDNA-1 | 62 | 23 | 128 |
| cfDNA-2 | 62 | 22 | 118 |

In the present disclosure, even when cancer cell lines and NA12878 are mixed at a ratio of 1:12,800, structural variant sequences are detected, and structural variant sequences are also sensitively identified in cfDNA of metastatic lung cancer patients. Therefore, by using the method for detecting structural variant sequences in cfDNA samples derived from cancer patients according to the present disclosure, patient-specific structural variant sequences can be detected with high sensitivity even when cancer-derived cfDNA exists in blood, and the like with low purity, and cancer cells remaining in a patient after cancer treatment can be detected even with a small sample, so that it can be universally utilized for the diagnosis of minimal residual disease regardless of cancer type.

The method according to one embodiment of the present disclosure described above may be implemented as a program (or application) and stored on a medium to be executed in combination with a hardware server.

The program described above may include codes coded in a computer language, such as C, C++, JAVA, or machine language, that can be read by the processor (CPU) of the computer through the device interface of the computer, so that the computer reads the program and executes the methods implemented as a program. Such codes may include functional codes related to functions that define functions necessary for executing the methods, and may include control codes related to execution procedures necessary for the processor of the computer to execute the functions according to a predetermined procedure. In addition, such codes may further include memory reference-related codes regarding which location (address) of the internal or external memory of the computer should be referenced for additional information or media necessary for the processor of the computer to execute the functions. In addition, if the processor of the computer needs to communicate with any other computer or server located remotely in order to execute the functions, the code may further include communication-related codes regarding how to communicate with any other computer or server located remotely using the communication module of the computer, what information or media to send and receive during communication, etc.

The storage medium means a medium that permanently stores data and can be read by a device, rather than a medium that stores data for a short period of time, such as a register, cache, or memory. Specifically, examples of the storage medium include, but are not limited to, ROM, RAM, CD-ROM, magnetic tape, floppy disk, or optical data storage device. That is, the program may be stored in various storage media on various servers that the computer can access, or in various storage media on the user's computer. In addition, the medium may be distributed to a computer system connected to a network, so that a computer-readable code may be stored in a distributed manner.

The steps of the method or algorithm described in connection with the embodiments of the present disclosure may be implemented directly in hardware, implemented as a software module executed by hardware, or implemented by a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or any other form of computer readable recording medium well known in the art to which the present disclosure pertains.

Although the embodiments of the present disclosure have been described above with reference to the attached drawings, those skilled in the art will understand that the present disclosure can be implemented in other specific forms without changing the technical spirit or essential characteristics thereof. Therefore, it should be understood that the embodiments described above are exemplary in all respects and not restrictive.

What is claimed is:

1. A method for detecting a structural variant sequence in a cfDNA sample derived from a cancer patient, the method performed by a processor of a device comprising:
1) obtaining consensus structural variant position data of a primary cancer sample that is commonly identified by analyzing a whole genome sequence (WGS) of a cancer tissue derived from a patient with two or more types of structural variant analysis software; and
2) matching a specific sequence generated by a structural variant existing in a cfDNA sample by matching a reference sequence of a structural variant position obtained in step 1) with a cfDNA WGS of the patient and by obtaining a structural variant sequence supporting read existing in the common structural variant position.

2. The method of claim 1, wherein a detection sensitivity is adjusted by adjusting a sequencing depth of the WGS.

3. The method of claim 1, wherein the WGS is at least two types selected from a group consisting of DELLY, BRASS, SvABA, dRanger, Pindell, BreakDancer, GASV, Hydra, CNVnator, and JuLI.

4. The method of claim 1, wherein step 1) comprises:
1-1) obtaining first structural variant position data of a primary cancer sample using one type of structural variant analysis software selected from a group consisting of DELLY, BRASS, SvABA, dRanger, Pindell, BreakDancer, GASV, Hydra, and CNVnator; and
1-2) obtaining the consensus structural variant position data of the primary cancer sample that is commonly identified by entering the first structural variant position data obtained in step 1-1) into JuLI.

5. The method of claim 1, wherein the cfDNA is derived from cerebrospinal fluid, pleural fluid, pericardial fluid, ascites, urine, whole blood, plasma or serum of the cancer patient.

6. The method of claim 1, wherein the cfDNA of the cancer patient in step 2) is obtained from the patient during or after cancer treatment.

7. The method of claim 1, wherein the structural variant is at least one type selected from a group consisting of duplication, deletion, transposition and insertion of a gene.

8. The method of claim 1, wherein the cancer is gastric cancer, lung cancer, non-small cell lung cancer, breast cancer, ovarian cancer, liver cancer, bronchial cancer, nasopharyngeal cancer, laryngeal cancer, pancreatic cancer, bladder cancer, colon cancer, colon cancer, cervical cancer, bone cancer, non-small cell bone cancer, blood cancer, skin cancer (melanoma, etc.), head or neck cancer, uterine cancer, rectal cancer, anal cancer, colon cancer, fallopian tube cancer, endometrial cancer, vaginal cancer, vulvar cancer, Hodgkin's disease, esophageal cancer, small intestine cancer, endocrine cancer, thyroid cancer, parathyroid cancer, adrenal cancer, soft tissue sarcoma, urethral cancer, penile cancer, prostate cancer, chronic or acute leukemia, lymphocytic lymphoma, kidney or ureteral cancer, renal cell carcinoma, renal pelvic carcinoma, polyploid carcinoma, salivary gland cancer, sarcoma, pseudomyxoma, hepatoblastoma, testicular cancer, glioblastoma, lip cancer, ovarian germ cell tumor, basal cell carcinoma, multiple myeloma, gallbladder cancer, choroidal melanoma, ampulla of Vater, peritoneal cancer, adrenal cancer, tongue cancer, small cell carcinoma, pediatric lymphoma, neuroblastoma, duodenal cancer, ureteral cancer, astrocytoma, meningioma, renal pelvis cancer, vulvar cancer, thymic cancer, central nervous system (CNS) tumor, primary central nervous system lymphoma, spinal cord tumor, brainstem glioma, or pituitary adenoma.

9. A method for providing information for minimal residual disease (MRD), the method performed by a processor of a device comprising:
1) obtaining consensus structural variant position data of a primary cancer sample that is commonly identified by analyzing a whole genome sequence (WGS) of a cancer tissue derived from a patient with two or more types of structural variant analysis software; and
2) matching a specific sequence generated by a structural variant existing in a cfDNA sample by matching a reference sequence of a structural variant position obtained in step 1) with a cfDNA WGS of the patient obtained from the patient after cancer treatment and by obtaining a structural variant sequence supporting read existing in the common structural variant position.

10. The method of claim 9, wherein the information for the minimal residual disease is information for the presence or absence of cancer cells remaining during or after treatment, a possibility of cancer recurrence, or a prognosis of cancer treatment.

11. The method of claim 9, wherein the treatment of step 2) is radiation therapy, immunotherapy, hormone therapy, chemotherapy, or surgical resection.
